(19) United States
(12) Patent Application Publication
(10) Pub. No.: US 2015/0189868 A1
(43) Pub. Date: Jul. 9, 2015

(54) BACTERICIDAL AND VIRUCIDAL COVERING MATERIAL AND METHOD FOR MAKING THE COVERING MATERIAL

(71) Applicants: Leopoldo Corsi, Ponsacco (IT); Luca Lombardi, Pistoia (IT); Solitario Nesti, Lamporecchio (IT)

(72) Inventors: Leopoldo Corsi, Ponsacco (IT); Luca Lombardi, Pistoia (IT); Solitario Nesti, Lamporecchio (IT)

(21) Appl. No.: 14/414,356

(22) PCT Filed: Jul. 16, 2012
(86) PCT No.: PCT/IT2012/000219

§ 371 Date: Jan. 12, 2015

(51) Int. Cl. A01N 25/10; A61L 2/08; A01N 59/16

(52) U.S. Cl. CPC A01N 25/10; A01N 59/16; A61L 2/088

(57) ABSTRACT

A method for making a bactericidal and virucidal covering material comprises the steps of preparing a web of thermoplastic polymeric material, applying a plurality of particles of photocatalytic material on the web of thermoplastic material and heating at least one portion of the web from a first, lower temperature to a second, upper temperature at least equal to the softening temperature of the thermoplastic polymeric material, in order to allow the particles of photocatalytic material to be integrated in the thermoplastic polymeric material.

### TECHNICAL FIELD

This invention relates to a method for making a bactericidal and virucidal covering material and to a web or slab of bactericidal and virucidal covering material made with the method.

More specifically, the invention relates to a bactericidal and virucidal material used for covering rooms that must be kept under special hygiene conditions in order to minimize risks of transmitting infections. For example, this invention is applicable in particular to furniture for hospital wards, medical rooms and operating rooms, which are subject to particularly stringent requirements regarding bacterial and viral charge reduction, or for other closed public spaces. The invention is also applicable in other contexts, such as food preparation and curing rooms, closed public spaces, means of transport for people, etc.

### BACKGROUND ART

As is known, hospital wards and medical rooms mist be kept under optimum hygiene conditions at all times in order to prevent the formation and propagation of bacteria and viruses which are harmful to the health of patients and medical operators.

Sanitization and disinfection are often carried out manually by personnel in charge of periodically cleaning all floor and wall surfaces with specific disinfecting substances.

Manual sanitization and disinfection procedures do not, however, guarantee the total elimination of pathogenic agents. Indeed, it should be borne in mind that manual cleaning, besides being time-consuming and labour intensive, is often inadequate for eliminating pathogenic micro-organisms in areas which are difficult to get at.

Also known in the prior art are devices, used mainly in operating rooms, for applying chemical sterilizing substances in the room to be sanitized.

These devices are activated automatically and allow the sterilizing substance to be sprayed in the operating room in order to sanitize the room thoroughly when it is not being used.

These devices, too, are not free of disadvantages, however.

In effect, the sterilizing substances used are harmful for operators, especially if applied in the large quantities needed for effective sanitization.

For that reason, after the sterilizing substance has been applied, the room treated cannot be used for a certain length of time, necessary for the sterilizing substance to be evacuated.

As a result, sanitizing and bacterial charge reduction procedures are very long and require the treated room to be left unused for some time after treatment. In other rooms, disinfection is carried out during routine cleaning procedures, whose efficacy is, generally speaking, very low.

In all these cases, however, the disinfecting action is temporary, that is to say, limited to the period of duration of the disinfection process. Once that period has passed (variable from a few minutes to several hours, depending on the disinfecting method used) contamination of a surface by any pathogenic micro-organism and the propagation of the micro-organism cease to be inhibited in any way.

### AIM OF THE INVENTION

In this context, the main technical purpose of this invention is to provide a covering material which allows the above mentioned disadvantages to be overcome and to provide also a method for making the material.

More specifically, this invention has for an aim to provide a covering material which can be used in any room, which allows any type of surface to be covered in order to eliminate the relative bacterial and viral charge efficiently and in a very limited time, and whose action is continuous over time.

A further aim of the invention is to provide a covering material which is inexpensive, and structurally simple and which allows the bacterial and viral charge to be eliminated without requiring any particular operations to be performed manually.

The technical purpose and aims specified are substantially achieved by a bactericidal and virucidal covering material comprising the technical features described in one or more of the accompanying claims from 17 to 23.

The technical purpose and aims specified are also achieved by a method for making the bactericidal and virucidal covering material, comprising the technical features described in one or more of the accompanying claims from 1 to 16.

### BRIEF DESCRIPTION OF THE DRAWINGS

Further features and advantages of the invention are more apparent in the non-limiting description which follows of a preferred and hence non-exclusive embodiment of a method for making a bactericidal and virucidal covering material and a bactericidal and virucidal covering material, as illustrated in the accompanying drawings, in which:

FIG. 1 is a schematic view of an operating room covered with the bactericidal and virucidal covering material according to this invention;

FIG. 2a is a schematic view of a roll made with the bactericidal and virucidal covering material according to the invention;

FIG. 2b is a schematic perspective view of a portion of the bactericidal and virucidal covering material according to this invention and shows in detail the layers making up the material;

FIGS. 3-9 are schematic layout views of installations which implement different embodiments of the method according to the invention.

### DETAILED DESCRIPTION OF THE PREFERRED EMBODIMENTS OF THE INVENTION

With reference to the accompanying drawings, a bactericidal and virucidal covering material according to this invention is denoted in its entirety by the numeral 1.

In the accompanying drawings (FIG. 2b), the thicknesses of the layers making up the material 1 are illustrated by way of example only and the thicknesses of the layers shown must not therefore be considered realistic.

The covering material 1 has a base layer 2 made from a thermoplastic polymeric material and designed to be connected to a surface to be covered, for example the floor or side wall of a medical room or the structure of hospital furniture or medical instruments (for example, a dental unit) or generic surfaces (for example, floors, walls and ceilings) of closed public spaces or means of transport.

Preferably, the base layer 2 has a certain degree of flexibility, allowing the covering material to adapt to the surface to be covered.

In other words, in a preferred embodiment, the covering material 1 is not self-supporting and may be easily rolled up in web form (also to incorporate a textile structure in order to enhance mechanical strength properties).

Alternatively, the covering material according to the invention (and in particular, the base layer 2) may be of a self-supporting type, provided it is mouldable, to form a covering body for a complex surface, such as, for example, a dental unit or the like (for example, in an application of this kind, a polymeric material such as ABS might be used).

In other terms, in a second embodiment, the base layer 2 might consist of materials having a defined stiffness so it can be conveniently used in specific applications.

On the side of it opposite the first surface 2a, the base layer 2 has a second surface 2b which is associated with a plurality of particles 3 of photocatalytic material.

According to the invention, the covering material 1 comprises a plurality of particles 3 of photocatalytic material laid over and at least partly integrated in the base layer 2.

Thus, the material substantially comprises a layer of particles 3 of photocatalytic material partly integrated (that is, embedded) in the base layer 2. In other words, there are no intermediate layers between the base layer 2 (made of thermoplastic polymeric material and the plurality of particles 3 of photocatalytic material. The covering material 1, on the other hand, has an intermediate coat 4 where the particles 3 of photocatalytic material and the base layer 2 are “merged” together in order to bond the two materials together.

Further, it should be noted that the particles 3 of photocatalytic material are positioned partly outside in view at the second surface 2b of the base layer 2. This enhances the efficacy of the photocatalytic action because it is not screened by other layers of material.

Preferably, the photocatalytic material consists of (a layer of) titanium dioxide.

More in detail, the layer of particles 3 of photocatalytic material consists of nanoparticle titanium dioxide.

Advantageously, the layer of particles 3 of photocatalytic material defines a nanometer thickness.

Preferably, the layer of particles 3 of photocatalytic material is obtained from a solution of nanoparticle titanium dioxide.

Still more preferably, the titanium dioxide solution is an aqueous solution.

According to preferred embodiments of the invention, the solution, (whether alcoholic or non-alcoholic), contains other chemical substances with an antimicrobial action.

Advantageously, by way of an example, one of the substances with an antimicrobial action is benzoic acid.

Preferably, the nanoparticle titanium dioxide is applied in the form of a colloidal dispersion where the liquid phase consists of water, solvent (amides, alcohols, acetates, ketones—preferably dimethylformamide) and a co-solvent (alcohols, acetates, ketones, aromatic hydrocarbons—preferably methyl ethyl ketone).

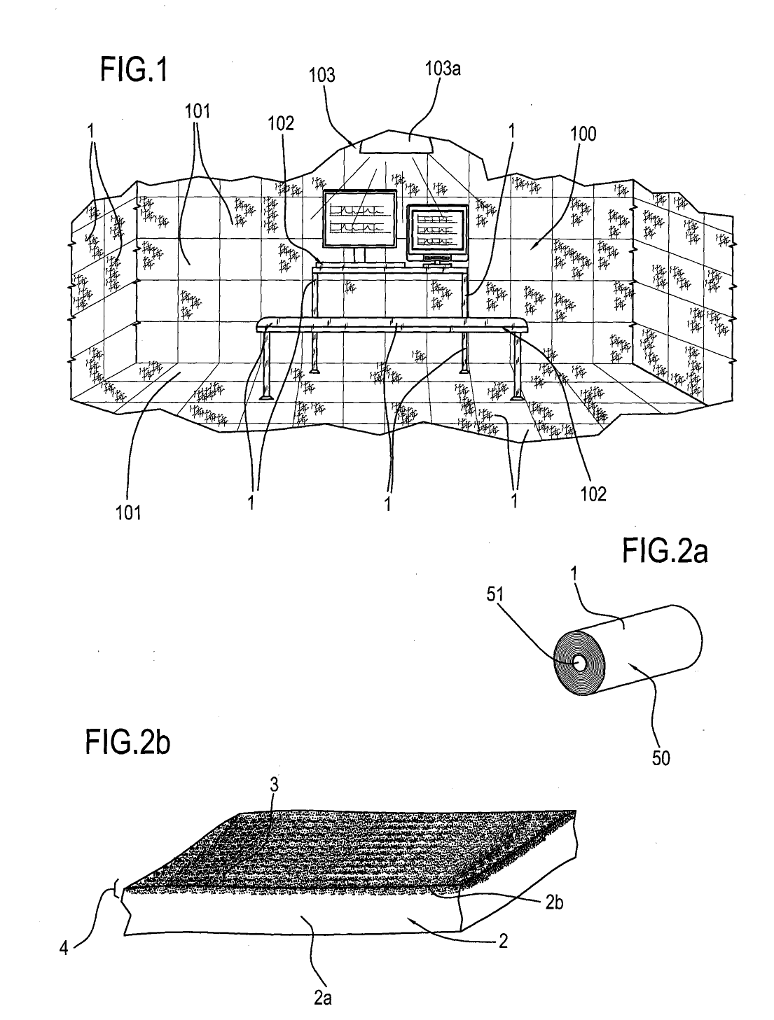

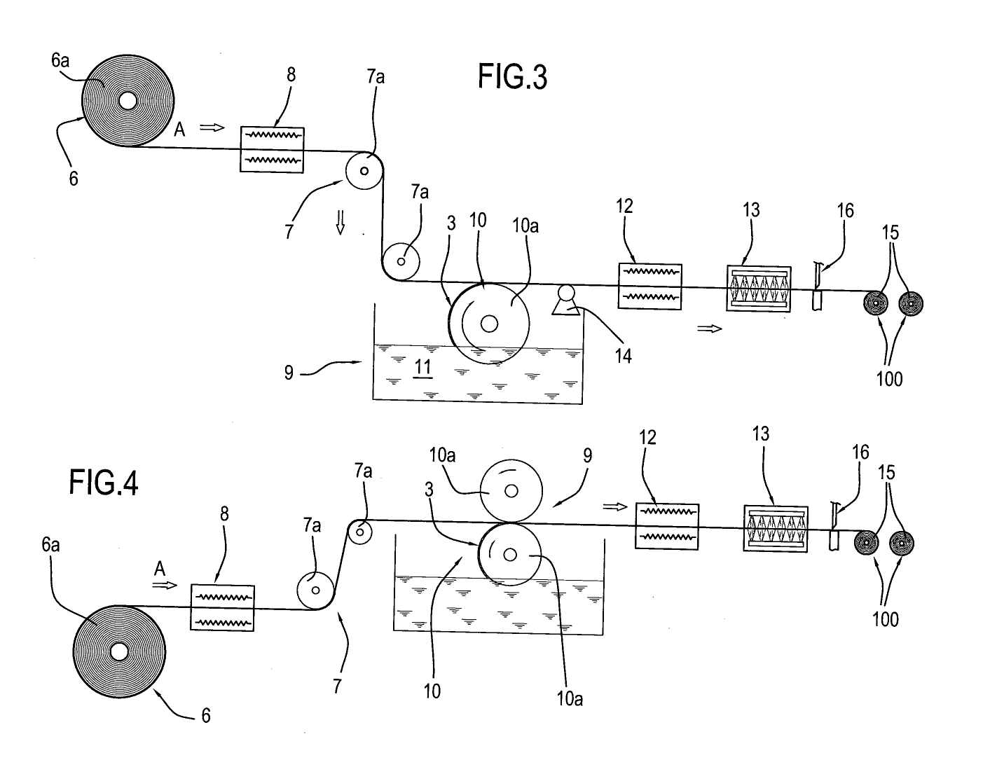

According to the first embodiment illustrated in FIG. 1, the anti-bacterial covering material 1 described below is used preferably to cover the walls 101 and the furniture 102 of an operating room 100.

Preferably, the material of the base layer 2 is chosen from one in the following list
Polyvinyl chloride (PVC)
Polyurethane (PU)
Polyester (PET)
Polypropylene (PP)
Acrylonitrile butadiene styrene (ABS)
Polycarbonate (PC)
Polyacrilates (PA)
Polymethacrylates (PA)
Polystyrene (PS)
Fluorinated polymers (PF)
and mixtures thereof.

This invention also has for an object a method for making the covering material 1 just described.

The method comprises a first step of preparing a web 6 or slab of thermoplastic polymeric material (preferably chosen from the above list).

The term “web” is used to specify that the thermoplastic material is not initially self-supporting and is wound in a roll.

Alternatively, however, as mentioned above, the thermoplastic material may have a structure which is substantially self-supporting but which may be moulded to define and cover surfaces with complex shapes (for example, ABS).

The web 6 is then advanced by unwinding and rewinding feed means 7 (and/or supports, preferably of the roller type 7a, or with chains and grippers for holding the web by its selvedges).

Next, at least one portion of the web is overheated from a first, lower (ambient) temperature to a second temperature at least equal to the softening temperature of the thermoplastic polymeric material.

The expression “softening temperature”, usually referred to as “Vicat Softening Temperature” (VST), is used in this text to denote the temperature region in which the polymer becomes progressively fluid. It is not a thermodynamic quantity but is of practical interest because it allows the material to be handled and treated even under high-viscosity conditions.

Softening points are measured according to precise ASTM standards, namely:
ASTM-D1525 (VST).
ASTM-E18 (ball and ring).
ASTM-D648 (heat deflection temperature, HDT).

For any given family of thermoplastics, both VST and HDT are influenced by plasticizers and fillers but usually fall within a temperature range between 20° C. and 400° C.

The table below shows, by way of example, the softening temperatures for some families of thermoplastics, that is VST (A: load 10N; B: load 50N) and HDT (A: stress 1.8 MPa; B: stress 0.46 MPa):

| Thermoplastic | VST A (° C.) | VST B (° C.) |
|---|---|---|
| PS | 82-106 | 78-101 |
| ABS | 93-125 | 83-116 |
| PVC | — | 81-127 |
| PE | 76-109 | 80 |
| PP | 135-162 | 45-128 |
| Thermoplastic | HDT A (° C.) | HDT B (° C.) |
| PC | 125-180 | 130-190 |
| Rigid PVC | 54-75 | 57-80 |
| PET | 70-240 | 72-250 |
| PMMA | 70-100 | 75-115 |
| PF | 45-160 | 70-260 |

Source: Michel Biron, Thermoplastics and Thermoplastic Composites: Technical Information for Plastics Users, Ed. Elsevier Science 2007

The step of overheating has a duration of between 30 and 300 seconds (preferably 150 seconds).

It should be noted that the step of overheating is preferably performed at a specific heating station 8 located along a feed direction “A” of the web 6.

In this regard, the method for making the material comprises a step of applying a plurality of particles of photocatalytic material to the web of thermoplastic material to be softened or already softened in order to embed a part of the particles in the web.

As mentioned above, the formulation (with particles of photocatalytic material) is defined by nanoparticle titanium dioxide which is applied in the form of colloidal dispersion.

The liquid phase of the colloidal dispersion consists of water, solvent (amides, alcohols, acetates, ketones—preferably dimethylformamide) and a co-solvent (alcohols, acetates, ketones, aromatic hydrocarbons—preferably methyl ethyl ketone).

The proportions (volumetric % v/v) vary from 5% (water)/95% (solvent+co-solvent) to 95% (water)/5% (solvent+co-solvent).

Preferably, the proportions are 10% (water)/90% (solvent+co-solvent). In the liquid phase, the proportions (volumetric) of solvent and co-solvent are variable from 50% each to 99% (solvent)−1% (co-solvent).

The formulation application process may be performed in different ways.

In a first embodiment, the application step comprises the following sub-steps (performed by suitable application means 9):

preparing a bath 11 comprising a formulation containing a predetermined percentage of particles 3 of photocatalytic material;

dipping at least part of a transfer means 10 into the bath 11 to collect defined portion of the formulation;

transporting a layer of photocatalytic material using the transfer means 10;

applying a plurality of the particles of photocatalytic material on the surface of the web by placing at least part of the transfer means 10 in contact with the web 6 of thermoplastic material (already softened or still to be softened).

In other words, application is performed by rotogravure or offset (transfer) printing.

The transfer means is preferably defined by at least one cylinder 10a partly dipped in the bath (and rotatable therein). In a preferred embodiment, the cylinder 10a is provided with micro cavities in order to collect a certain quantity of formulation.

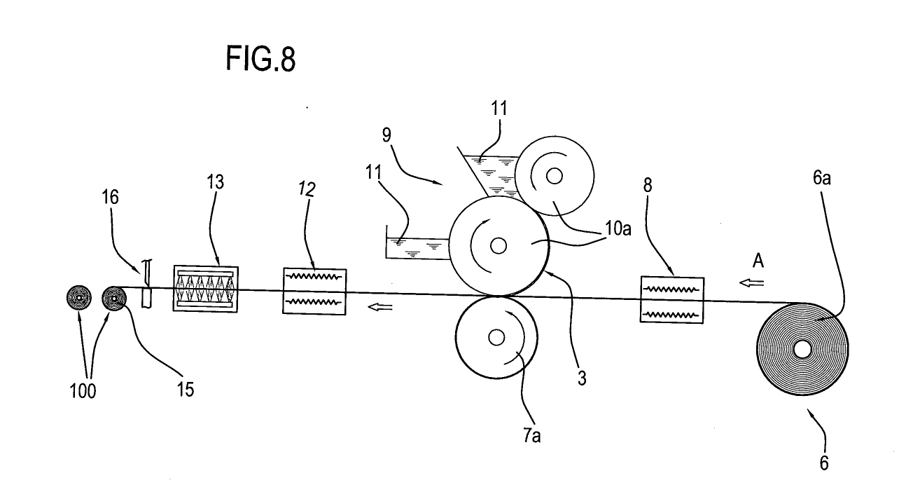

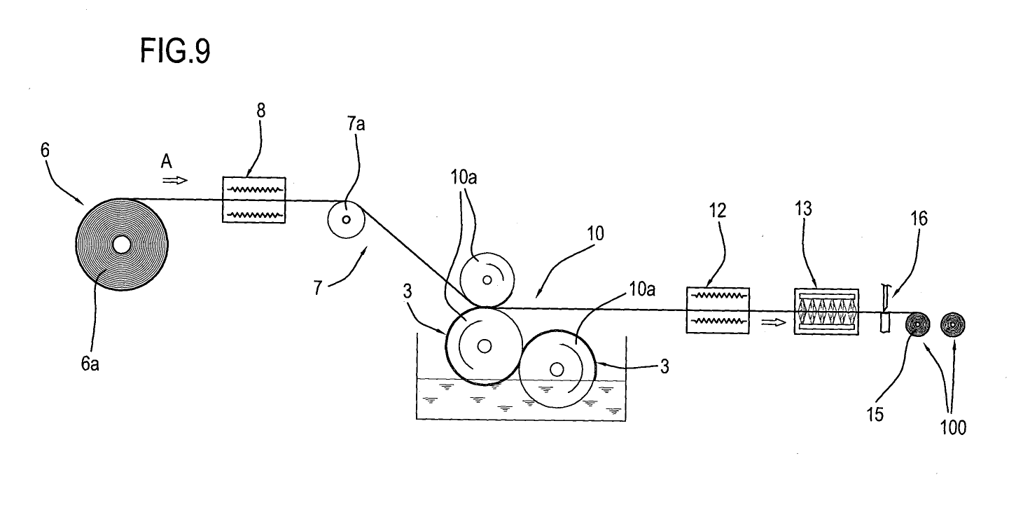

There might also be more than one cylinder 10a, counter-rotating relative to one another, to allow optimum adjustment of the quantity of formulation, or equipped with doctor blades (FIG. 8) to remove the excess material and leave only a defined quantity of product.

In an alternative embodiment, the application step is performed by spraying the photocatalytic material onto the web (in one or more passes).

In a further embodiment, the application step is performed by heat transfer from a medium (paper or polymeric film) on which there is a layer of particles 3 of photocatalytic material which is transferred to the web 6 and made to adhere thereto by heating and compression applied by hot cylinders.

With reference to the accompanying drawings, attention is drawn to the fact that the feed means 7, and more specifically, the rollers 7a, are configured (and designed) to not interfere with the side of the web 6 covered with the particles 3 of photocatalytic material (at least until performing a step of stabilizing the bond between the web 6 and the particles 3, which is described in more detail below).

It should be noted that by applying the formulation after the heating step (when the polymeric material of the web 6 is “softened”), the particles of photocatalytic material become physically integrated (embedded) in the web 6, forming the intermediate coat 4 described above.

Alternatively, the heating step may be performed after the formulation (that is, the particles 3 of photocatalytic material) has been applied so that embedding occurs at a later stage and more gradually.

in a more complete embodiment, the heating step is performed both before and after applying the formulation.

In other words, in this embodiment, the heating step comprises a first heating step performed before the application step, in such a way as to soften the thermoplastic polymeric material, and a second heating step performed after the application step in order to improve the action of embedding and fixing the particles of photocatalytic material in the web of thermoplastic polymeric material, stabilizing their adhesion.

To speed up production, the method preferably comprises a step of stabilizing the bond between the web 6 of thermoplastic polymeric material and the particles 3 of photocatalytic material after the application step.

In a first embodiment, the stabilizing step comprises a step of cooling the web 6 performed after the application step.

In the embodiments illustrated, the cooling step is of the “forced” type, that is to say, it is carried out by blowing means 13.

Alternatively (or in addition), the stabilizing step comprises a step of calendering the web 6, also after the application step.

This process (calendering) is performed in machines (calenders) consisting of parallel-axis rollers, set at an adjustable distance from each other, and rotating at low speed. It involves making the web 6 of polymeric material in the softened state pass between the pairs of rollers (as in metal rolling) to obtain sheets or slabs or webs of desired thickness and in which the particles 3 of photocatalytic material are integrated.

It should be noted that the calendering cylinders are adjustable in temperature in order to improve their effect.

Preferably, there is a step of adjusting the amount of particles of photocatalytic material (that is, of the formulation) applied or to be applied to the web.

In a first embodiment, the adjustment step comprises, before the application step, a sub-step of metering the quantity of particles of photocatalytic material to be applied to the web.

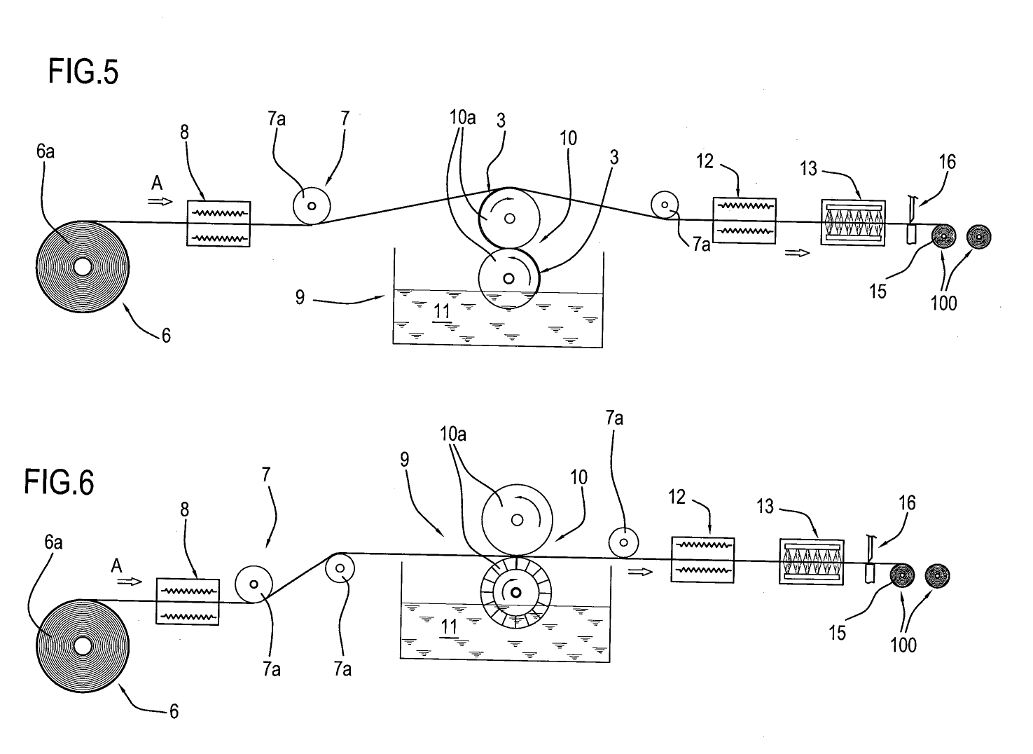

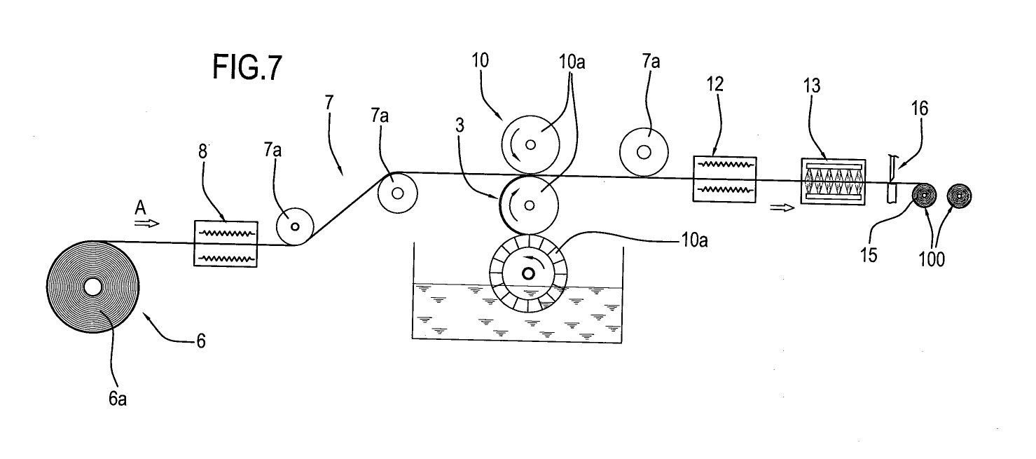

This step may be performed using counter-rotating rollers (FIG. 5) or the aforementioned doctor blades (FIG. 8).

Alternatively (or in addition), the adjustment step comprises a sub-step of removing the excess particles 3 of photocatalytic material, using scraping/adjusting means 14, after the application step.

Preferably, as stated, the covering material is wound into rolls of flexible material and thus, the method comprises a final step of rolling up the web of thermoplastic polymeric material to form a roll 50 of anti-bacterial covering material after the application and fixing step.

This step is performed preferably using rolling-up means 15 which are associated preferably with suitable cutting means 16 configured to adjust the final length of the roll 50 (provided with a supporting central core 51) of covering material 1.

It should be noted that the bactericidal action of the covering material is activated by subjecting the material to irradiation by a light source 103.

With reference to FIG. 1, it should be noted that the medical room 100 (operating room) comprises the light source 103 for catalyzing the bactericidal covering material 1.

Advantageously, the light source 103 is a lamp 103a with an emission spectrum of between 290 and 780 nm to irradiate the bactericidal covering material 1.

Preferably, the power of the lamp 103a is 250 W, with a maximum radiation of 380 nm.

In use, the bactericidal covering material 1, placed on the walls 101 and furniture 102 of the operating room 100 is catalyzed by the lamp 103a. The lamp 103a thus activates the bactericidal and virucidal property of the titanium dioxide contained in the material 1. The bactericidal and virucidal property therefore depends on the length of time the material 1 is irradiated.

Below, by way of example, are the results of experiments conducted to demonstrate bacterial reduction efficacy over irradiation time, in particular for reducing the charge of the bacterium Staphylococcus aureus.

Experimentally, the bactericidal property was measured according to ISO 27447:2009 (AATCC 100:2004 for the preparation of inoculants).

REDUCTION RATE (%) MEASURED 24 h AFTER IRRADIATION

| | Bacterial charge | | |
|---|---|---|---|
| | (UCF/25 cm2) | | Reduction |
| | | after | rate |
| Sample | time zero | 24 h at 37° C. | (%) |
| PVC as is | 2,100,000 | 2,300,000 | 0 |
| Anti-bacterial PVC | 4,000,000 | 1,100 | 99.9 |
| (1 pass: formulation 1-0.5-8.5) | | | |
| Anti-bacterial PVC | 4,500,000 | 3,200 | 99.9 |
| (1 pass: formulation 1-0.5-7.5) | | | |
| Anti-bacterial PVC | 2,100,000 | 2,300 | 99.9 |
| (2 pass: formulation 1-0.5-7.5) | | | |
| Anti-bacterial PVC | 2,200,000 | 4,000 | 99.8 |
| (3 pass: formulation 1-0.5-7.5) | | | |

Analysis of the Staphylococcus aureus inoculant after 24 h confirmed that the surface has a bactericidal effect.

The invention achieves the above mentioned aims and has important advantages.

In effect, the production of a covering material which is polymer based but fully adaptable to the surface to be covered considerably extends the range of uses of the material, making it possible to cover entire medical rooms, from partition walls to furniture.

Further, the integration of the photocatalytic particles into the base layer by softening the latter facilitates and speeds up the process for the production of the material, making the process at once more economical and efficient.

Moreover, the possibility of winding the covering material into rolls facilitates its storage and transportation, making the product easily available and sellable in any part of the world, with limited transport costs.

Moreover, it should be considered that the material is activated quickly and automatically by the light source. Thus, the room need not be manually sanitised by personnel in charge, with obvious advantages in terms of time and labour.

1. A method for making a bactericidal and virucidal covering material characterized in that it comprises the steps of:
preparing a web of thermoplastic polymeric material;
applying a plurality of particles of photocatalytic material on the web of thermoplastic material;
heating at least one portion of the web from a first, lower temperature to a second, upper temperature at least equal to the softening temperature of the thermoplastic polymeric material, in order to allow the particles of photocatalytic material to be integrated in the thermoplastic polymeric material.

2. The method according to claim 1, wherein the heating step is performed before the step of applying a plurality of particles of photocatalytic material.

3. The method according to claim 1, wherein the heating step is performed at least partly after the step of applying a plurality of particles of photocatalytic material.

4. The method according to claim 1, wherein the heating step is divided into:
a first heating step performed before the application step, in such a way as to soften the thermoplastic polymeric material;
a second heating step performed after the application step in order to improve the action of embedding the particles of photocatalytic material in the web of thermoplastic polymeric material, stabilizing their adhesion.

5. The method according to claim 1, wherein it comprises a step of stabilizing the bond between the thermoplastic polymeric material and the particles of photocatalytic material after the application step.

6. The method according to claim 5, wherein the stabilizing step comprises a step of cooling the web after the application step.

7. The method according to claim 5, wherein the stabilizing step comprises a step of calendering the web after the application step.

8. The method according to claim 1, wherein it comprises a step of adjusting a quantity of particles of photocatalytic material applied or to be applied to the web.

9. The method according to claim 8, wherein the adjustment step comprises, before the application step, a sub-step of metering the quantity of particles of photocatalytic material to be applied to the web.

10. The method according to claim 8, wherein the adjustment step comprises, after the application step, a sub-step of removing the excess particles of photocatalytic material.

11. The method according to claim 1, wherein the application step comprises the following sub-steps:
preparing a bath comprising a formulation containing a predetermined percentage of particles of photocatalytic material;
dipping at least part of a transfer means into the bath to collect a portion of the formulation;
transporting a layer of formulation using the transfer means;
placing at least part of the transfer means in contact with the web of thermoplastic material in order to apply the formulation containing a percentage of particles of photocatalytic material.

12. The method according to claim 1, wherein the application step is performed by spraying a formulation containing a predetermined percentage of particles of photocatalytic material onto the web.

13. The method according to claim 1, wherein it comprises a step of rolling up the web of thermoplastic polymeric material after the application step, in order to form a roll of anti-bacterial covering material.

14. The method according to claim 1, wherein the thermoplastic polymeric material is chosen from the following list of families of materials:
Polyvinyl chloride (PVC),
Polyurethane (PU),
Polyester (PET),
Polypropylene (PP),
Acrylonitrile butadiene styrene (ABS),
Polycarbonate (PC),
Polyacrilates (PA),
Polymethacrylates (PMA),
Polystyrene (PS),
Fluorinated polymers (PF),
and mixtures thereof.

15. The method according to claim 1, wherein the photocatalytic material is titanium dioxide.

16. The method according to claim 1, wherein the photocatalytic material is nanoparticle titanium dioxide.

17. A bactericidal and virucidal covering material in web form, comprising:
a base layer made of a thermoplastic polymeric material;
a plurality of particles of photocatalytic material partly embedded in the base layer and facing the outside thereof.

18. The covering material according to claim 17, wherein the plurality of particles of photocatalytic material is defined by a uniform layer of the particles partly embedded in the base layer.

19. The covering material according to claim 17, wherein the base layer of thermoplastic polymeric material has a non-self-supporting structure.

20. The covering material according to claim 13, wherein the base layer of thermoplastic polymeric material has a rigid, self-supporting structure.

21. The covering material according to claim 17, wherein the thermoplastic polymeric material is chosen from the following list of families of materials:
Polyvinyl chloride (PVC),
Polyurethane (PU),
Polyester (PET),
Polypropylene (PP),
Acrylonitrile butadiene styrene (ABS),
Polycarbonate (PC),
Polyacrilates (PA),
Polymethacrylates (PMA),
Polystyrene (PS),
Fluorinated polymers (PF),
and mixtures thereof.

22. The covering material according to claim 17, wherein the photocatalytic material is titanium dioxide.

23. The covering material according to claim 17, wherein the photocatalytic material is nanoparticle titanium dioxide.

24. A roll of bactericidal and virucidal covering material, comprising:
a central core;
a web made of an anti-bacterial covering material according to claim 13, wound around the central core.

25. Use of a bactericidal and virucidal covering material according to claim 17 to at least partly cover surfaces of rooms or items of furniture in a medical room, wherein the covering material is photo-activated by a lamp located in the medical room.
